(19) United States
(12) Patent Application Publication
(10) Pub. No.: US 2021/0059532 A1
(43) Pub. Date: Mar. 4, 2021

(54) TREATMENT SUPPORT APPARATUS AND DETERMINATION METHOD IN TREATMENT SUPPORT APPARATUS

(71) Applicant: Shimadzu Corporation, Kyoto (JP)

(72) Inventor: Hiroyuki TSUMATORI, Kyoto (JP)

(21) Appl. No.: 16/919,921

(22) Filed: Jul. 2, 2020

(30) Foreign Application Priority Data
Aug. 27, 2019 (JP) 2019-155040

(51) Int. Cl. A61B 5/00; A61N 5/06

(52) U.S. Cl. CPC A61B 5/0071; A61B 5/0082; A61N 2005/0626; A61B 5/742; A61N 5/062

(57) ABSTRACT

This treatment support apparatus is provided with a fluorescence detection unit for detecting fluorescence emitted from a photosensitizer by being irradiating with treatment light, and a determination unit for determining a cancer region based on a temporal change of a signal value of the fluorescence detected by the fluorescence detection unit.

### CROSS-REFERENCE TO RELATED APPLICATIONS

The related application number JP2019-155040, entitled “Treatment Support Apparatus and Determination Method in Treatment Support Apparatus”, filed on Aug. 27, 2019, invented by Hiroyuki Tsumatori, upon which this patent application is based, is hereby incorporated by reference.

### BACKGROUND OF THE INVENTION

### Field of the Invention

The present invention relates to a treatment support apparatus and a determination method in the treatment support apparatus.

### Description of the Background Art

Conventionally, a cancer treatment method is known in which a photosensitizer accumulated on a cancer treatment target site of a subject is irradiated with treatment light. Such a method is disclosed, for example, in Japanese Unexamined Patent Application Publication No. 2017-71654.

In the above-described Japanese Unexamined Patent Application Publication No. 2017-71654, as a cancer treatment method, a photoimmunotherapy using near-infrared light (treatment light) is disclosed. In this photoimmunotherapy, a chemical agent in which a substance (photosensitizer) that emits fluorescence by absorbing near-infrared light and an antibody that selectively binds to a cancer cell are bound is administered to a subject. Then, for the purpose of circulating the chemical agent throughout the subject, the subject is left for a period of, e.g., one day (24 hours) from the administration of the chemical agent. During this period, the antibody of the chemical agent administered to the subject selectively binds to a cancer cell. Then, the subject is irradiated with near-infrared light as treatment light. As a result, the cancer cell is killed by the heat generated from the substance which emits fluorescence of the chemical agent.

In the above-mentioned Japanese Unexamined Patent Application Publication No. 2017-71654, IRDye (registered trademark) 700Dx is used as a substance that emits fluorescence.

### SUMMARY OF THE INVENTION

It is an object of the present invention to provide a treatment support apparatus capable of accurately determining a cancer region when performing cancer treatment performed by irradiating a photosensitizer as described above with treatment light and a determination method using the treatment support apparatus.

In order to achieve the above-described object, as a result of the intensive study, the inventor of the present application has obtained new findings that in cases where fluorescence emitted from a photosensitizer by being irradiated with treatment light is a cancer-derived fluorescence, a signal value obtained from this fluorescence behaves characteristically with the lapse of time, and in cases where fluorescence emitted from a photosensitizer by being irradiated with treatment light is non-cancer-derived fluorescence, a signal value obtained from this fluorescence does not substantially change with the lapse of time.

The treatment support apparatus according to the first aspect of the present invention determines a cancer region using the new findings. That is, the treatment support apparatus according to the first aspect of the present invention is a treatment support apparatus for supporting cancer treatment by imaging a cancer treatment target site of a subject when performing the cancer treatment by irradiating a photosensitizer accumulated in the cancer treatment target site with treatment light, wherein the treatment support apparatus includes a fluorescence detection unit configured to detect fluorescence emitted from the photosensitizer by being irradiated with the treatment light and a determination unit configured to determine a cancer region based on a temporal change of a signal value of the fluorescence detected by the fluorescence detection unit.

In the treatment support apparatus according to the first aspect of the present invention, since the apparatus is configured as described above, by utilizing the findings that in cases where the fluorescence detected by the fluorescence detection unit is cancer-derived fluorescence, the temporal change of the signal value of the fluorescence appears with a certain magnitude, on the other hand, in cases where the fluorescence detected by the fluorescence detection unit is non-cancer-derived fluorescence, the temporal change of the signal value of the fluorescence is sufficiently small (almost none), it is possible to distinguish between a cancer-derived fluorescence and a non-cancer-derived fluorescence. As a result, a cancer region and a non-cancer region can be distinguished from each other by the above-described determination unit. With this, it is possible to provide a treatment support apparatus capable of accurately determining a cancer region when performing cancer treatment by irradiating photosensitizer with treatment light.

In the treatment support apparatus according to the first aspect of the present invention, preferably, the determination unit is configured to determine that a region is a cancer region when a temporal change value of the signal value of the fluorescence detected by the fluorescence detection unit is equal to or greater than a threshold value.

With this configuration, when the fluorescence detected by the fluorescence detection unit is cancer-derived fluorescence, it is possible to easily and accurately determine the cancer region indicating the temporal change value equal to or greater than the threshold value by utilizing that the temporal change of the signal value of the fluorescence is large.

In this case, preferably, the determination unit is configured to determine that a region is a non-cancer region when a temporal change value of the signal value of the fluorescence detected by the fluorescence detection unit is less than the threshold value.

With this configuration, when the fluorescence detected by the fluorescence detection unit is non-cancer-derived fluorescence, it is possible to easily and accurately determine the non-cancer region indicating the temporal change value less than the threshold value by utilizing the fact that the there is almost no temporal change of the signal value of the fluorescence.

In the treatment support apparatus according to the first aspect of the present invention, preferably, the determination unit is configured to determine the cancer region based on the temporal change of the signal value of the fluorescence detected by the fluorescence detection unit in a predetermined period after initiation of irradiation of the treatment light.

With this configuration, the determination of the cancer region can be performed at an early stage, so that the determination result of the cancer region can be obtained at an early stage to use for the treatment at an early stage.

In the treatment support apparatus according to the first aspect of the present invention, preferably, the treatment support apparatus further includes an estimation unit configured to estimate a finish time of treatment of the cancer treatment target site by the treatment light based on the temporal change of the signal value of the fluorescence in the cancer region detected by the fluorescence detection unit in a predetermined period after initiation of irradiation of the treatment light.

With this configuration, when the fluorescence detected by the fluorescence detection unit is cancer-derived fluorescence, it is possible to predict the temporal change of the signal value of the cancer-derived fluorescence and estimate the time when the treatment of the cancer treatment target site by the treatment light is finished by using the fact that the signal value of the fluorescence includes a characteristic temporal change. As a result, the physician in charge of the treatment can grasp the time when the treatment of the cancer treatment target site by the treatment light is finished based on the actual treatment effect. With this, unlike a case in which a subject is irradiated with treatment light for a predetermined period, it is possible to suppress unnecessarily continued irradiation of the subject with the treatment light in the treatment, and it is also possible to suppress a shortage of irradiation time of the treatment light to the subject in the treatment. As a result, the treatment of the cancer treatment target site by treatment light can be performed at a required and sufficient treatment time to sufficiently secure the treatment effect by the treatment light.

In this case, preferably, the estimation unit is configured to acquire a function representing the temporal change of the signal value of the fluorescence in the cancer region based on the temporal change of the signal value of the fluorescence in the cancer region detected by the fluorescence detection unit in the predetermined period and estimate the finish time of the treatment of the cancer treatment target site by the treatment light based on the acquired function.

With this configuration, it is possible to easily and accurately estimate the time when the treatment of the cancer treatment target site by the treatment light is finished based on the function representing the characteristic temporal change of the signal value of the cancer-derived fluorescence.

In the configuration for acquiring the above-described function, preferably, the function is a function representing a waveform having two peaks. Note that the present inventor has obtained a new finding that in cases where the fluorescence detected by the fluorescence detection unit is cancer-derived fluorescence, the temporal change of the signal value of the fluorescence may be represented by a waveform having two peaks. Thus, by configuring as described above, in cases where the signal value obtained from the cancer-derived fluorescence is represented by a waveform having two peaks, it is possible to acquire a function that more accurately represents the characteristic temporal change of the signal value of the cancer-derived fluorescence. As a result, it is possible to more accurately estimate the time when the treatment of the cancer treatment target site by the treatment light is finished based on the acquired function.

In this case, preferably, the function includes a function representing a decay curve corresponding to the temporal change of the signal value of the fluorescence in the cancer region detected by the fluorescence detection unit.

Note that the inventor of the present application has obtained a new finding that in cases where the temporal change of the signal value of the cancer-derived fluorescence is represented by a waveform having two peaks, the temporal change of the signal value of the cancer-derived fluorescence can be accurately represented by a function representing a decay curve of a Double-Boltzmann function or the like. Thus, by configuring as described above, in cases where the signal value obtained from the cancer-derived fluorescence is represented by a waveform having two peaks, it is possible to acquire a function representing a decay curve that more accurately represents the characteristic temporal change of the signal value of the cancer-derived fluorescence. As a result, the time when the treatment of the cancer treatment target site by the treatment light is finished can be estimated more accurately based on the acquired function representing the decay curve.

In the treatment support apparatus according to the first aspect of the present invention, preferably, the determination unit is configured to determine whether to complete the treatment of the cancer treatment target site by the treatment light based on the temporal change of the signal value of the fluorescence in the cancer region detected by the fluorescence detection unit.

With this configuration, it is possible to determine whether to complete the treatment of the cancer treatment target site by the treatment light based on the actual treatment effect. As a result, unlike the case in which it is determined whether to complete the treatment of the cancer treatment target site by the treatment light based only on the irradiation time of the treatment light, the treatment of the cancer treatment target site by the treatment light can be completed in a state in which the treatment effect by the treatment light has been sufficiently secured.

In the treatment support apparatus according to the first aspect of the present invention, preferably, a fluorescent image is generated based on the fluorescence detected by the fluorescence detection unit, and the generated fluorescent image is output to a display unit.

With this configuration, the physician in charge of the treatment can perform the treatment of the cancer treatment target site by the treatment light while confirming the fluorescent image showing the cancer treatment target site displayed on the display unit. As a result, the physician can easily perform the treatment of the cancer treatment target site by the treatment light.

A determination method in a treatment support apparatus according to a second aspect of the present invention is a determination method in a treatment support apparatus for supporting cancer treatment by imaging a cancer treatment target site of a subject when performing the cancer treatment by irradiating a photosensitizer accumulated in the cancer treatment target site with treatment light. The method includes the steps of: detecting fluorescence emitted from the photosensitizer by being irradiated with the treatment light; and determining a cancer region based on a temporal change of a signal value of the detected fluorescence.

In the determination method in the treatment support apparatus according to the second aspect of the present invention, in the same manner as in the treatment support apparatus according to the first aspect of the present invention, it is possible to provide a determination method in the treatment support apparatus capable of accurately determining a cancer region when performing the cancer treatment by irradiating the photosensitizer with the treatment light.

### BRIEF DESCRIPTION OF THE DRAWINGS

FIG. 1 is a schematic diagram of a treatment support system equipped with a treatment support apparatus according to an embodiment of the present invention.

FIG. 2 is a perspective view of a treatment support apparatus according to an embodiment of the present invention.

FIG. 3 is a schematic diagram of an imaging unit of a treatment support apparatus according to an embodiment of the present invention.

FIG. 4 is a schematic diagram of an interior of an imaging unit of a treatment support apparatus according to an embodiment of the present invention.

FIG. 5 is a schematic diagram of an entire configuration of a treatment support system equipped with a treatment support apparatus according to an embodiment of the present invention.

FIG. 6A is a schematic diagram of an image generated by a treatment support apparatus according to an embodiment of the present invention and is a schematic diagram of a fluorescent image.

FIG. 6B is a schematic diagram of an image generated by a treatment support apparatus according to an embodiment of the present invention and is a schematic diagram of a visible light image.

FIG. 6C is a schematic diagram of a composite image in which a fluorescent image and a visible light image are composed.

FIG. 7A is a graph showing a temporal change of a signal value obtained from fluorescence.

FIG. 7B is a schematic diagram for explaining a determination of a cancer region and an estimation of a treatment finish time based on a temporal change of a signal value obtained from a cancer-derived fluorescence.

FIG. 7C is a schematic diagram for explaining a determination of completion of treatment based on a temporal change of a signal value obtained from a cancer-derived fluorescence.

FIG. 8 is a schematic diagram of a treatment support system equipped with a treatment support apparatus according to a first modification of an embodiment of the present invention.

FIG. 9 is a schematic diagram of a treatment support system equipped with a treatment support apparatus according to a second modification of an embodiment of the present invention.

### DESCRIPTION OF THE PREFERRED EMBODIMENTS

Hereinafter, some embodiments in which the present invention is embodied will be described with reference to the drawings.

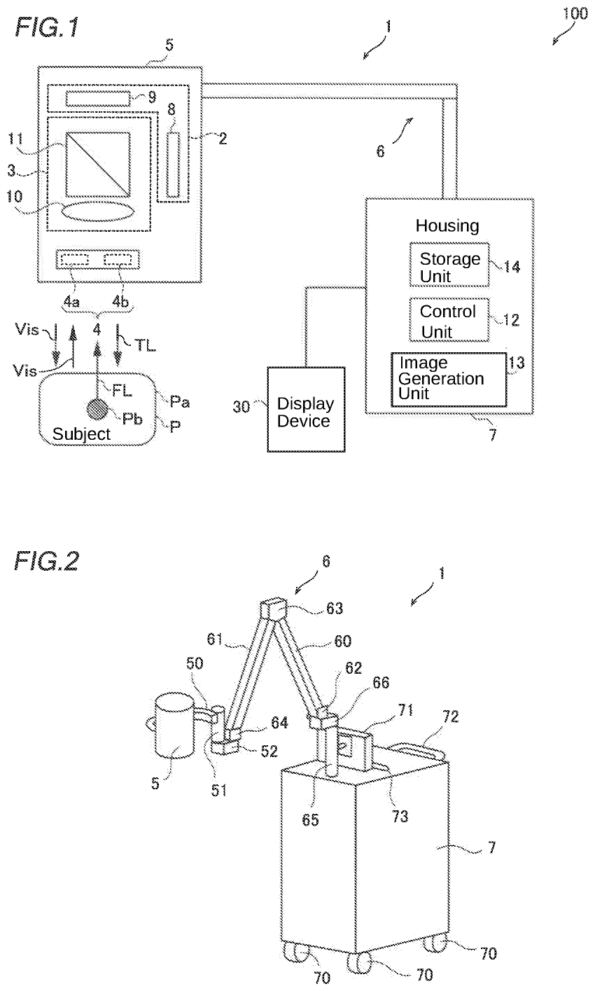

Referring to FIG. 1 to FIG. 7, a configuration of a treatment support system 100 equipped with a treatment support apparatus 1 according to an embodiment of the present invention will be described.

### (Configuration of Treatment Support System)

As shown in FIG. 1, the treatment support system 100 according to one embodiment is provided with a treatment support apparatus 1 and a display device 30. The treatment support apparatus 1 is a treatment support apparatus for supporting cancer treatment by imaging a cancer treatment target site Pa when performing the cancer treatment by irradiating a photosensitizer Pb accumulated in the cancer treatment target site Pa in a subject P with treatment light TL.

The subject P is, for example, a human, a dog, a cat, etc. The cancer treatment target site Pa is, for example, a mouth, a throat, a chest, a gastrointestinal tract, a liver, an adrenal gland, etc.

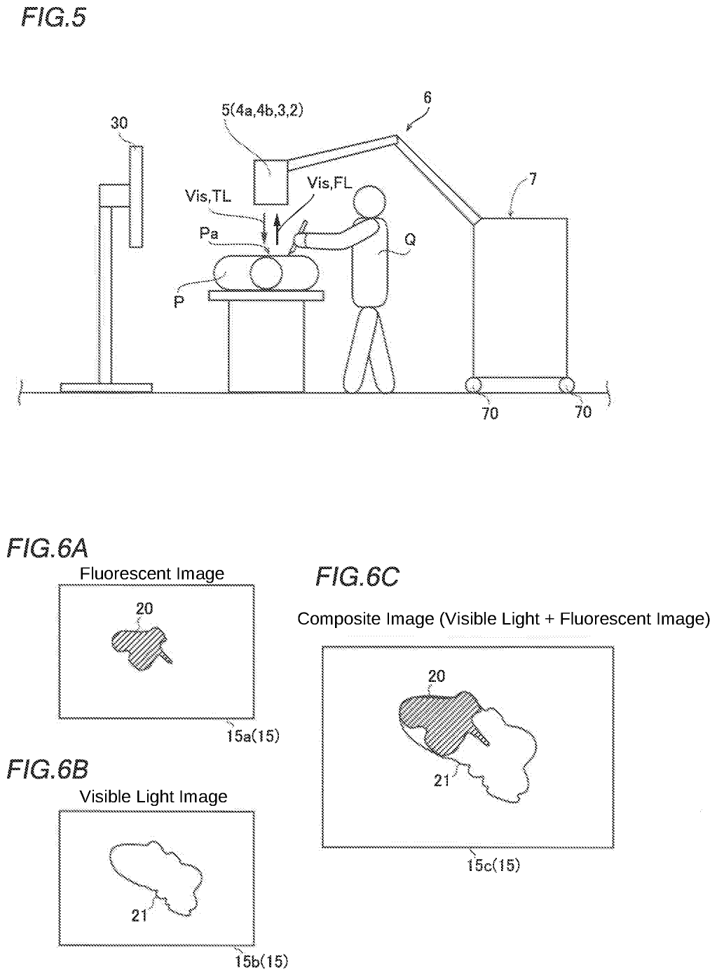

The treatment support apparatus 1 is configured to detect fluorescence FL emitted from a photosensitizer Pb by being irradiated with the treatment light TL and visualize the cancer 20 (epithelial cancer, see FIG. 6) to thereby assist a surgical operation of a surgeon Q (see FIG. 5). The detailed configuration of the treatment support apparatus 1 will be described later.

The display device 30 is configured to display a captured image 15 of a treatment target site Pa output from the treatment support apparatus 1 (see FIG. 6). The display device 30 is, for example, a monitor such as a liquid crystal display. The display device 30 is an example of the “display unit” recited in claims.

### (Configuration of Treatment Support Apparatus)

As shown in FIG. 1, the treatment support apparatus 1 according to one embodiment is provided with an imaging unit 5 including a light receiving unit 2, an optical system 3, and a light source unit 4, an arm mechanism 6, and a housing 7.

The light receiving unit 2 includes a visible light detection unit 8 and a fluorescence detection unit 9. The visible light detection unit 8 is configured to detect visible light Vis. The fluorescence detection unit 9 is configured to detect fluorescence FL. The detailed configurations of the visible light detection unit 8 and the fluorescence detection unit 9 will be described later.

The optical system 3 includes a zoom lens 10 and a prism 11. The optical system 3 is configured to perform the separation of the visible light Vis reflected from a subject P and the fluorescence FL emitted from a photosensitizer Pb by being irradiated with the treatment light TL. The detailed configuration of the optical system 3 will be described later.

The light source unit 4 is provided with a visible light source 4a for emitting visible light Vis to a subject P and a treatment light source 4b for emitting treatment light TL for exciting electronics of the photosensitizer Pb that have been applied in the body of the subject P toward the subject P to emit fluorescence FL. The visible light source 4a and the treatment light source 4b include, for example, a light emitting diode (LED). The photosensitizer Pb is, for example, IRDye (registered trademark) 700Dx (hereinafter referred to as “IR700”). The IR700 is a substance that emits fluorescence FL by absorbing treatment light TL as near-infrared light.

Here, a near-infrared light immunotherapy (MR-PIT) as a cancer treatment method will be described. In a near-infrared light immunotherapy, first, a chemical agent in which a photosensitizer Pb (IR700 or the like) that emits fluorescence FL by absorbing treatment light TL and an antibody (antibody of an epidermal growth factor receptor, etc.) that selectively binds to the cancer 20 (see FIG. 6) are combined is administered to a subject P by infusion or the like. Then, for the purpose of circulating the chemical agent throughout the subject P, the subject P is left for a period of, e.g., one day (24 hours) from the administration of the chemical agent. During this period, the antibody of the chemical agent administered to the subject P selectively binds to the cancer 20. As a result, the photosensitizer Pb is sufficiently accumulated in the cancer treatment target site Pa. The subject P is then irradiated with treatment light TL. As a result, it is considered that the cancer 20 is killed by the action of the heat generated from the photosensitizer Pb of the chemical agent.

The housing 7 includes a control unit 12, an image generation unit 13, and a storage unit 14. The housing 7 is, for example, a cart in which a PC (personal computer) is mounted. The control unit 12 is configured to control irradiation of light (visible light Vis, treatment light TL) from the light source unit 4, suspension of the irradiation, etc., based on an input operation by an operation unit (not shown). Note that the control unit 12 is composed of, for example, a processor such as a CPU (Central Processing Unit). The control unit 12 is an example of the “determination unit” and the “estimation unit” recited in claims.

The image generation unit 13 is configured to generate a fluorescent image 15a (see FIG. 6) as a captured image 15 based on the signal of the fluorescence FL detected by the fluorescence detection unit 9. The image generation unit 13 is configured to generate a visible light image 15b (see FIG. 6) as a captured image 15 based on the signal of the visible light Vis detected by the visible light detection unit 8.

The image generation unit 13 is configured to generate a composite image 15c (see FIG. 6) as a captured image 15 in which the fluorescent image 15a and the visible light image 15b are composed. The image generation unit 13 includes a processor, such as, e.g., a GPU (Graphics Processing Unit) and an FPGA (Field-Programmable Gate Array) configured for image-processing. The storage unit 14 is configured to store, for example, the captured image 15 generated by the image generation unit 13. The storage unit 14 includes a nonvolatile memory, a hard disk drive (HDD), etc.

As shown in FIG. 2, the housing 7 includes a plurality (four) of wheels 70, an arm mechanism 6 provided near the front of the housing 7 on the upper surface of the housing 7, an imaging unit 5 provided on the arm mechanism 6 via a sub arm 50, and a monitor 71. A handle 72 used to move the housing 7 is provided at the rear portion of the housing 7. A recess 73 for mounting an operation unit (not shown) used for a remote operation of the treatment support apparatus 1 is formed on the upper surface of the housing 7.

The arm mechanism 6 is provided on the anterior side of the housing 7 (opposite side of the handle 72). The arm mechanism 6 is provided with a first arm member 60 connected by a hinge portion 62 to a support portion 66 arranged on a columnar support 65 provided on the front side of the housing 7. The first arm member 60 is configured to be swingable with respect to the housing 7 about the hinge portion 62 via the columnar support 65 and the support portion 66. A monitor 71 is mounted on the columnar support 65.

A second arm member 61 is connected to the upper end of the first arm member 60 by a hinge portion 63. The second arm member 61 is configured to be swingable with respect to the first arm member 60 via the hinge portion 63. Therefore, the first arm member 60 and the second arm member 61 can be freely adjusted in angle.

At the bottom of the second arm member 61, a support portion 52 is connected by a hinge portion 64. The support portion 52 is configured to be swingable with respect to the second arm member 61 via the hinge portion 64. The support portion 52 is provided with a rotating shaft 51. A sub arm 50 supporting the imaging unit 5 is rotatable about the rotating shaft 51 provided at the distal end of the second arm member 61. Therefore, the imaging unit 5 moves between the position on the front side of the housing 7 with respect to the arm mechanism 6 and the position on the rear side of the housing 7 (handle 72 side) with respect to the arm mechanism 6 which is in a posture when the housing 7 is moved, by the rotation of the sub arm 50.

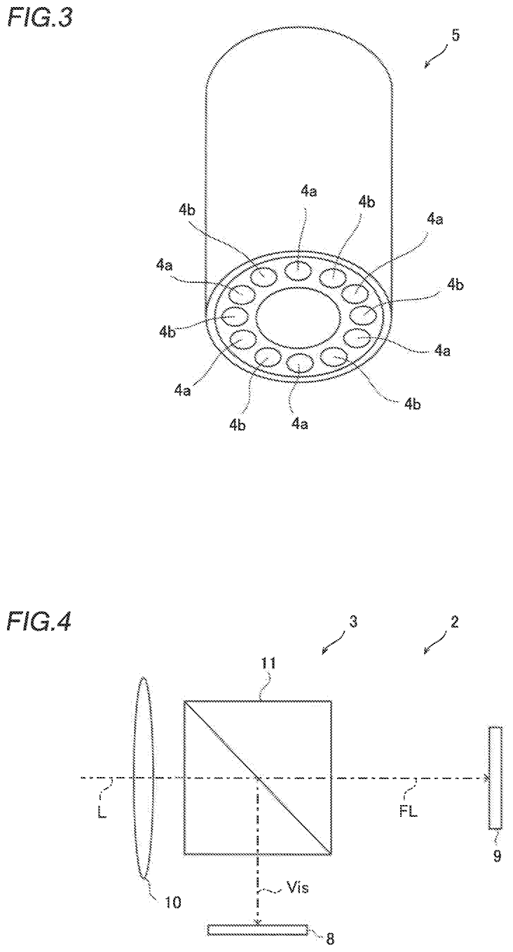

As shown in FIG. 3, the imaging unit 5 contains a light receiving unit 2, an optical system 3, and a light source unit 4. In FIG. 3, the visible light source 4a and the treatment light source 4b are each composed of six LEDs. The visible light source 4a is configured to emit, e.g., white light as visible light Vis toward a subject P. The treatment light source 4b is configured to emit, e.g., near-infrared light toward a subject P as treatment light TL for exciting electronics of a photosensitizer Pb to emit fluorescence FL.

As shown in FIG. 4, the light receiving unit 2 is provided with a visible light detection unit 8 and a fluorescence detection unit 9. The optical system 3 is provided with a zoom lens 10 that is reciprocally moved along the optical axis L by a lens movement mechanism (not shown) for focusing and a prism 11. The prism 11 is configured to perform separation of the visible light Vis reflected from a subject P and the fluorescence FL emitted from a photosensitizer Pb by being irradiated with the treatment light TL.

The visible light detection unit 8 is configured to detect the visible light Vis emitted from the visible light source 4a and reflected from a subject P. The fluorescence detection unit 9 is configured to detect the fluorescence FL emitted from the photosensitizer Pb administered to the body of the subject P by the treatment light TL emitted from the treatment light source 4b. The visible light detection unit 8 and the fluorescence detection unit 9 include, for example, an image sensor such as a CMOS (Complementary Netal Oxide Semiconductor) image sensor and a CCD (Charge Coupled Device) image sensor. It should be noted that as the visible light detection unit 8, a visible light detection unit capable of acquiring a visible light image 15b as a color image is used.

The visible light detection unit 8 and the fluorescence detection unit 9 are configured to detect the visible light Vis and the fluorescence FL by a common optical system 3 when detecting the visible light Vis and the fluorescence FL. Specifically, the visible light Vis and the fluorescence FL entered the zoom lens 10 along the optical axis L pass through the zoom lens 10 before reaching the prism 11. Of the visible light Vis and the fluorescence FL that have reached the prism 11, the visible light Vis is reflected by the prism 11 and reaches the visible light detection unit 8. Of the visible light Vis and the fluorescence FL that have reached the prism 11, the fluorescence FL passes through the prism 11 and reaches the fluorescence detection unit 9. Note that the reflected light of the treatment light TL from the subject P is reflected by the prism 11. Therefore, the reflected light of the treatment light TL from the subject P is prevented from reaching the fluorescence detection unit 9.

As shown in FIG. 5, the treatment support system 100 is configured as a treatment support system for performing a treatment support by acquiring the fluorescent image 15a and the visible light image 15b (see FIG. 6) of the subject P when performing the cancer treatment and displaying them on the display device 30. The treatment support apparatus 1 is configured to image the treatment target site Pa of the subject P from the outside of the subject P when the surgeon Q performs the cancer surgical operation of the subject P. Specifically, the photosensitizer Pb inside the subject P generates the fluorescence FL by the treatment light TL irradiated from the treatment light source 4b provided in the treatment support apparatus 1. The fluorescence detection unit 9 provided in the treatment support apparatus 1 detects the fluorescence FL generated from the photosensitizer Pb inside the subject P.

As shown in FIG. 6A to FIG. 6C, the treatment support apparatus 1 is configured to image the tissue 21 including the cancer 20 of the subject P. The treatment support apparatus 1 is configured to generate a composite image 15c by composing the fluorescent image 15a in which the cancer 20 is reflected and the visible light image 15b in which the tissue 21 is reflected, and output the fluorescent image 15a, the visible light image 15b, and the composite image 15c to the display device 30. The display device 30 is configured to display the fluorescent image 15a, the visible light image 15b, and the composite image 15c, respectively.

### (Behavior of Signal Value of Fluorescence)

When the subject P is irradiated with the treatment light TL, the chemical agent containing the photosensitizer Pb is circulating throughout the subject P, and the treatment light TL is emitted not only to the cancer cell but also to the periphery of the cancer cell, so that when the subject P is irradiated with the treatment light TL, not only the chemical agent selectively bonded to the cancer cell but also the chemical agent in the periphery of the cancer cell emit the fluorescence FL. Therefore, since the cancer-derived fluorescence FL and the non-cancer-derived fluorescence FL are detected at a time, the cancer-derived fluorescence FL and the non-cancer-derived fluorescence FL cannot be distinguished from each other by, for example, simply seeing the image in which the fluorescence FL is reflected. Therefore, the cancer region cannot be accurately determined from the detected fluorescence FL.

However, new findings have been obtained. That is, in cases where fluorescence FL emitted from a photosensitizer Pb by being irradiated with the treatment light TL is caser-derived fluorescence FL, the signal value obtained from the fluorescence FL behaves characteristically with the lapse of time. On the other hand, in cases where fluorescence FL emitted from a photosensitizer Pb by being irradiated with the treatment light TL is non-cancer-derived fluorescence FL, the signal value obtained from the non-cancer-derived fluorescence FL does not substantially change or decreases moderately monotonously with the lapse of time or, and exhibits a change that clearly differs from the signal value obtained from the cancer-derived fluorescence FL.

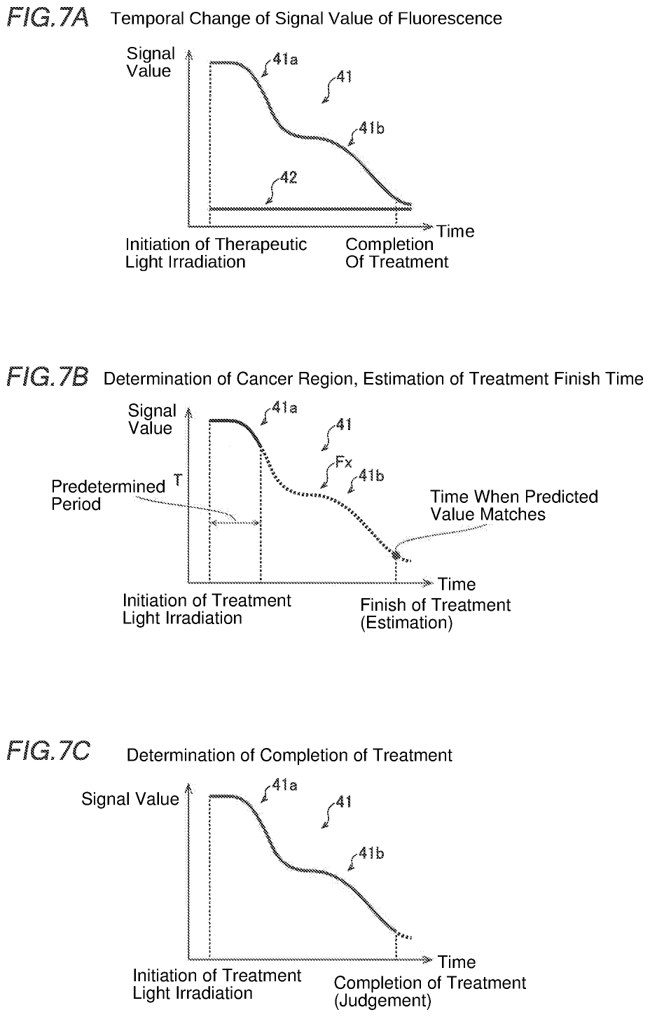

Now, referring to FIG. 7A, the behavior of the signal value obtained from the cancer-derived fluorescence FL and the behavior of the signal value obtained from the non-cancer-derived fluorescence FL will be described. FIG. 7A is a graph schematically showing the temporal change of the signal value obtained from the fluorescence FL during the period from the initiation of irradiating the subject P with the treatment light TL to the completion of the treatment of the treatment target site Pa. As shown in FIG. 7A, the cancer signal waveform 41 showing the temporal change of the signal value of the cancer-derived fluorescence FL behaves characteristically with the lapse of time.

In particular, the cancer signal waveform 41 behaves such that the value (strength) decays gradually with the lapse of time while generating two peaks of a first peak 41a and a second peak 41b. The first peak 41a is a first peak that occurred first and is a peak that occurred at the early stage of the irradiation period of the treatment light TL. The second peak 41b is a second peak that occurred after the first peak 41a and is a peak that occurred after the mid-stage of the irradiation period of the treatment light TL. On the other hand, the non-cancer signal waveform 42 representing the temporal change of the signal value of the non-cancer-derived fluorescence FL behaves to exhibit a substantially constant value regardless of the laps of time. It is considered that this is because in the chemical agent not bonded to the cancer 20, the photosensitizer Pb continues to emit light without being decomposed, while in the chemical agent bonded to the cancer 20, the photosensitizer Pb is decomposed and quenched when the cancer 20 is killed.

As described above, since the temporal change of the signal value obtained from the cancer-derived fluorescence FL and the temporal change of the signal value obtained from the non-cancer-derived fluorescence FL differ greatly, the cancer region (cancer-derived fluorescence FL) can be easily determined based on the temporal change of the signal value of the fluorescence FL detected by the fluorescence detection unit 9.

### (Configuration for Determination of Cancer Region)

Therefore, in this embodiment, the control unit 12 determines (detects) the cancer region based on the temporal change of the signal value of the fluorescence FL detected by the fluorescence detection unit 9. The control unit 12 determines that the region is a cancer region when the temporal change value (temporal change rate, temporal change difference, etc.) of the signal value of the fluorescence FL detected by the fluorescence detection unit 9 is equal to or greater than a threshold value. The control unit 12 determines that the region is a non-cancer region when the temporal change value of the signal value of the fluorescence FL detected by the fluorescence detection unit 9 is less than a threshold value. The threshold value is a value determined in advance by experiments or the like.

For example, the control unit 12 determines whether or not the temporal change value of the signal value of the detected fluorescence FL is equal to or greater than a threshold value for each pixel of the fluorescence detection unit 9. The control unit 12 determines that a pixel in which the temporal change value of the signal value of the detected fluorescence FL is equal to or greater than a threshold value is a pixel constituting a cancer region (i.e., a pixel in which cancer-derived fluorescence FL is detected). Then, the control unit 12 determines that the pixel group determined to constitute a cancer region is a cancer region. In addition, the control unit 12 determines that a pixel in which the temporal change value of the signal value of the detected fluorescence FL is less than a threshold value is a pixel constituting a non-cancer region (i.e., a pixel in which non-cancer-derived fluorescence FL is detected). Then, the control unit 12 determines that the pixel group determined to constitute a non-cancer detection is a non-cancer region. Note that the determination may be performed not only for each pixel but also for each pixel block comprising a plurality of pixels.

Further, as shown in FIG. 7B, the control unit 12 determines the cancer region based on the temporal change of the signal value of the fluorescence FL detected by the fluorescence detection unit 9 during a predetermined period T after initiation of irradiation of the subject P with treatment light TL. The control unit 12 acquires the temporal change value of the signal value of the fluorescence FL detected by the fluorescence detection unit 9 during the predetermined period T, and determines the cancer region based on the acquired temporal change value. The predetermined period T is an early stage of the irradiation period of the treatment light TL. For example, the predetermined period T is a duration from the initiation of irradiation of the subject P with the treatment light TL until the temporal change of the signal value begins to grow (until the temporal change value of the signal value becomes equal to or greater than a predetermined value).

Further, for example, the predetermined period T is a period from the initiation of irradiation of the subject P with the treatment light TL until a first peak 41a of the cancer signal waveform 41 occurs. In other words, the predetermined period T is a period from the initiation of irradiation of the subject P with the treatment light TL until the signal value starts decreasing. The predetermined period T is a value determined in advance by experiments or the like. Note that in FIG. 7B, for easy understanding, the temporal change of the signal value of the fluorescence FL which has been detected by the fluorescence detection unit 9 is indicated by a solid line, and the temporal change of the signal value of the fluorescence FL which has not yet been detected by the fluorescence detection unit 9 is virtually indicated by a broken line.

### (Configuration of Estimation of Treatment Finish Time)

Further, in this embodiment, the control unit 12 estimates (acquires) the time when the treatment of the cancer treatment target site Pa by the treatment light TL is finished (completed) based on the temporal change of the signal value of the fluorescence FL in the cancer region detected by the fluorescence detection unit 9 during the predetermined period T after the initiation of irradiation of the subject P with the treatment light Tl. The predetermined period T for estimating the treatment finish time and the predetermined period T for determining the cancer region may differ from each other.

The control unit 12 acquires a function Fx representing the temporal change of the signal value of the fluorescence FL in the cancer region based on the temporal change of the signal value of the fluorescence FL in the cancer region detected by the fluorescence detection unit 9 during the predetermined period T. The function Fx is a function representing a waveform (i.e., cancer signal waveform 41) having two peaks. The function Fx includes a Double-Boltzmann function which is a function representing a decay curve corresponding to the temporal change of the signal value of the fluorescence FL in the cancer region detected by the fluorescence detection unit 9.

Acquiring the temporal change of the signal value of the fluorescence FL in the cancer region detected by the fluorescence detection unit 9 during the predetermined period T shown in FIG. 7B, the control unit 12 performs curve fitting by a Double-Boltzmann function to specify a function Fx approximating the acquired temporal change. The control unit 12 estimates the time when the treatment of the cancer treatment target site Pa by the treatment light TL is finished based on the acquired function Fx. Note that a Double-Boltzmann function is an example of a “function representing a decay curve” recited in claims.

Specifically, the control unit 12 acquires, based on the function Fx, the time when the predicted value relating to the signal value of the fluorescence FL matches a predetermined value, and estimates the acquired time as the time when the treatment of the cancer treatment target site Pa by the treatment light TL is finished. The predicted value relating the signal value of the fluorescence FL is, for example, the temporal change value of the signal value of the fluorescence FL or the signal value of the fluorescence FL. For example, the control unit 12 acquires, based on the function Fx, the time when the temporal change value (predicted value) of the signal value of the fluorescence FL after the second peak 41b of the cancer signal waveform 41 occurs matches the predetermined temporal change value (predetermined value). Then, the control unit 12 estimates the acquired time as the time when the treatment of the cancer treatment target site Pa by the treatment light TL is finished. Note that the predetermined temporal change value is a value determined in advance by experiments or the like.

Further, for example, the control unit 12 acquires the time when the signal value (predicted value) of the fluorescence FL matches a predetermined signal value (predetermined value) based on the function Fx. Then, the control unit 12 estimates the acquired time as the time when the treatment of the cancer treatment target site Pa by the treatment light TL is finished. The predetermined signal value can be obtained based on, for example, the signal value of the fluorescence FL in the cancer region detected by the fluorescence detection unit 9. Specifically, the predetermined signal value can be obtained as a value obtained by multiplying the maximum value of the signal value of the fluorescence FL in the cancer region detected by the fluorescence detection unit 9 by a predetermined ratio. The predetermined ratio is a value determined in advance by experiments or the like.

Note that in cases where the cancer region is composed of a pixel group, it is considered that there is a variation in the temporal change of the signal value of the fluorescence FL for each pixel. In this case, for example, the treatment finish time may be estimated for a typical value, such as, e.g., a mean value or a median value, of the signal value of the fluorescence FL for each pixel. Further note that, for example, the treatment finish time may be estimated for the sum of signal values of fluorescence FL of all the pixels. Further note that, for example, the treatment finish time may be estimated for each signal value of the fluorescence FL for each pixel, and the latest time among the times acquired for each pixel may be acquired as a treatment finish time.

Further, the control unit 12 performs control of notifying the user (doctor) of the estimated time when the treatment of the cancer treatment target site Pa by the treatment light TL is finished by, for example, displaying the time on the display device 30.

### (Configuration for Determination of Completion of Treatment)

In this embodiment, as shown in FIG. 7C, the control unit 12 determines whether to complete the treatment of the cancer treatment target site Pa by the fluorescence FL based on the temporal change of the signal value of the fluorescence FL in the cancer region detected by the fluorescence detection unit 9 at the end period (after the occurrence of the second peak 41b of the cancer signal waveform 41) of the irradiation period of the treatment light TL. In cases where the value relating to the signal value of the fluorescence FL detected by the fluorescence detection unit 9 does not coincide with the predetermined value (until the value coincides), the control unit 12 determines that the treatment of the cancer treatment target site Pa by the treatment light TL is not completed (the treatment has not been completed). When the value relating to the signal value of the fluorescence FL detected by the fluorescence detection unit 9 matches the predetermined value, the control unit 12 determines that the treatment of the cancer treatment target site Pa by the treatment light TL is completed (the treatment has been completed).

The value relating to the signal value of the fluorescence FL detected by the fluorescence detection unit 9 is, for example, the temporal change value of the signal value of the fluorescence FL detected by the fluorescence detection unit 9 or the signal value of the fluorescence FL detected by the fluorescence detection unit 9, which are predetermined signal values. For example, the control unit 12 determines whether to complete the treatment of the cancer treatment target site Pa by the treatment light TL based on whether or not the temporal change value of the signal value of the fluorescence FL detected by the fluorescence detection unit 9 matches a predetermined temporal change value. The predetermined temporal change value is a value determined in advance by experiments or the like.

Further, for example, the control unit 12 determines whether to complete the treatment of the cancer treatment target site Pa by the treatment light TL based on whether or not the signal value of the fluorescence FL detected by the fluorescence detection unit 9 matches a predetermined signal value. The predetermined signal value can be obtained based on, for example, the signal value of the fluorescence FL in the cancer region detected by the fluorescence detection unit 9. Specifically, the predetermined signal value can be obtained as a value obtained by multiplying the maximum value of the signal value of the fluorescence FL in the cancer region detected by the fluorescence detection unit 9 by a predetermined ratio. The predetermined ratio is a value determined in advance by an experiment or the like.

Also in the case of determining the treatment completion, similarly to the case of estimating the treatment finish time, in cases where the cancer region is composed of a pixel group, it is considered that there is a variation in the temporal change of the signal value of the fluorescence FL for each pixel. In this case, for example, the determination of the treatment completion may be performed for a typical value, such as, e.g., a mean value and a median value, of the signal value of the fluorescence FL for each pixel. Further, for example, the determination of the treatment completion may be performed for the sum of the signal values of the fluorescence FL of all the pixels. In addition, for example, the above-described determination of the treatment completion may be performed for each signal value of the fluorescence FL for each pixel, and when it is determined to be the treatment completion in all the pixels, it may be determined that the treatment has been completed.

Further, the control unit 12 performs control to notify the user (doctor) that the treatment of the cancer treatment target site Pa by the treatment light TL has been completed by, for example, making a sound or displaying a notification on the display device 30. At this time, for example, the control unit 12 controls the treatment light source 4b of the light source unit 4 so as to stop the irradiation of the treatment light TL. In addition, for example, the control unit 12 performs control to notify the user (doctor) that the irradiation of the treatment light TL should be terminated.

### (Configuration of Fluorescent Image)

The image generation unit 13 generates a fluorescent image 15a capable of visually distinguishing between the cancer region and the non-cancer region based on the determination result of the cancer region by the control unit 12. For example, the image generation unit 13 generates a fluorescent image 15a capable of visually distinguishing between the cancer region and the non-cancer region by displaying the cancer region and the non-cancer region in different colors. Also, for example, the image generation unit 13 generates a fluorescent image 15a capable of visually distinguishing between the cancer region and the non-cancer region by highlighting the contour of the cancer region.

### Effects of Embodiment

In this embodiment, the following effects can be obtained.

In this embodiment, as described above, the control unit 12 is configured to determine the cancer region based on the temporal change of the signal value of the fluorescence FL detected by the fluorescence detection unit 9.

With this, in cases where the fluorescence FL detected by the fluorescence detection unit 9 is the cancer-derived fluorescence FL, the temporal change of the signal value of the fluorescence FL appears with a certain degree of magnitude. On the other hand, in cases where the fluorescence FL detected by the fluorescence detection unit 9 is the non-cancer-derived fluorescence FL, the cancer-derived fluorescence FL and the non-cancer-derived fluorescence FL can be distinguished by using that the temporal change of the signal value of the fluorescence FL is sufficiently small (almost none). As a result, the cancer region and the non-cancer region can be distinguished by the control unit 12. As a result, it is possible to provide the treatment support apparatus 1 capable of accurately determining the cancer region when performing the cancer treatment performed by irradiating the photosensitizer Pb with the treatment light TL.

Further, in this embodiment, as described above, the control unit 12 is configured to determine that the region is a cancer region in cases where the temporal change value of the signal value of the fluorescence FL detected by the fluorescence detection unit 9 is equal to or greater than the threshold value. Thus, in cases where the fluorescence FL detected by the fluorescence detection unit 9 is the cancer-derived fluorescence FL, it is possible to easily and accurately determine the cancer region representing the temporal change value equal to or greater than the threshold value by utilizing the fact that the temporal change of the signal value of the fluorescence FL is large.

Further, in this embodiment, as described above, the control unit 12 is configured to determine that the region is a non-cancer region in cases where the temporal change value of the signal value of the fluorescence FL detected by the fluorescence detection unit 9 is less than the threshold value. Thereby, in cases where the fluorescence FL detected by the fluorescence detection unit 9 is non-cancer-derived fluorescence FL, it is possible to easily and accurately determine the non-cancer region representing the temporal change value less than the threshold value by utilizing that there is almost no temporal change of the signal value of the fluorescence FL.

Further, in this embodiment, as described above, the control unit 12 is configured to determine the cancer region based on the temporal change of the signal value of the fluorescence FL detected by the fluorescence detection unit 9 during the predetermined period T after the initiation of irradiation of the treatment light TL. As a result, the determination of the cancer region can be performed at an early stage, so that the determination result of the cancer region can be obtained at an early stage and can be used for the treatment at an early stage.

Further, in this embodiment, as described above, the control unit 12 is configured to estimate the treatment finish time of the cancer treatment target site Pa by the treatment light TL based on the temporal change of the signal value of the fluorescence FL in the cancer region detected by the fluorescence detection unit 9 during the predetermined period T after the initiation of irradiation of the treatment light TL.

Thus, in cases where the fluorescence FL detected by the fluorescence detection unit 9 is cancer-derived fluorescence FL, it is possible to predict the temporal change of the signal value of the cancer-derived fluorescence FL and estimate the time when the treatment of the cancer treatment target site Pa by the treatment light TL is finished by using that there is a characteristic temporal change of the signal value of the fluorescence FL. As a result, the physician in charge of the treatment can know the time when the treatment of the cancer treatment target site Pa by the treatment light TL based on the actual treatment effect is finished.

As a result, unlike the case in which the subject P is irradiated with the treatment light TL for a predetermined period, it is possible to suppress unnecessary continuous irradiation of the subject P with the treatment light TL for the treatment, and it is also possible to suppress insufficient irradiation time of the treatment light TL to the subject P for the treatment. As a result, the treatment of the cancer treatment target site Pa by the treatment light TL can be performed at a required and sufficient treatment time capable of sufficiently securing the treatment effect by the treatment light TL.

Further, in this embodiment, as described above, the control unit 12 is configured to acquire the function Fx representing the temporal change of the signal value of the fluorescence FL in the cancer region based on the temporal change of the signal value of the fluorescence FL in the cancer region detected by the fluorescence detection unit 9 in the predetermined period T, and estimate the time when the treatment of the cancer treatment target site Pa by the treatment light TL is finished based on the acquired function Fx. As a result, it is possible to easily and accurately estimate the time when the treatment of the cancer treatment target site Pa by the treatment light TL is finished based on the function Fx representing the characteristic temporal change of the signal value of the cancer-derived fluorescence FL.

In this embodiment, as described above, the function Fx is configured to be a function representing a waveform having two peaks. Note that the present inventor has obtained a new finding that in cases where the fluorescence FL detected by the fluorescence detection unit 9 is cancer-derived fluorescence FL, there is a case in which the temporal change of the signal value of the fluorescence FL is represented by a waveform having two peaks. Thus, by configuring as described above, in cases where the signal value obtained from cancer-derived fluorescence FL is represented by a waveform having two peaks, it is possible to acquire a function Fx that more accurately represents the characteristic temporal change of the signal value of the cancer-derived fluorescence FL. As a result, the time when the treatment of the cancer treatment target site Pa by the treatment light TL is finished can be estimated more accurately based on the acquired function Fx.

Further, in this embodiment, as described above, the function Fx is configured to include a Double-Boltzmann function corresponding to the temporal change of the signal value of the fluorescence FL in the cancer region detected by the fluorescence detection unit 9. Note that the present inventor has obtained a new finding that in cases where the temporal change of the signal value of the cancer-derived fluorescence FL is represented by a waveform having two peaks, the temporal change of the signal value of the cancer-derived fluorescence FL can be accurately represented by a Double-Boltzmann function.

Thus, by configuring as described above, in cases where the signal value obtained from the cancer-derived fluorescence FL is represented by a waveform having two peaks, it is possible to obtain a Double-Boltzmann function Fx that more accurately represents the characteristic temporal change of the signal value of the cancer-derived fluorescence FL. As a result, the time when the treatment of the cancer treatment target site Pa by the treatment light TL is finished can be estimated more accurately based on the acquired Double-Boltzmann function Fx.

Further, in this embodiment, as described above, the control unit 12 is configured to determine whether to complete the treatment of the cancer treatment target site Pa by the treatment light TL based on the temporal change of the signal value of the fluorescence FL in the cancer region detected by the fluorescence detection unit 9. Thereby, it can be determined whether to complete the treatment of the cancer treatment target site Pa by the treatment light TL based on the actual treatment effect. As a result, unlike the case in which it is determined whether to complete the treatment of the cancer treatment target site Pa by the treatment light TL based only on the irradiation time of the treatment light TL, the treatment of the cancer treatment target site Pa by the treatment light TL can be completed in a state in which the treatment effect by the treatment light TL is sufficiently secured.

Further, in this embodiment, as described above, the fluorescent image 15a is generated based on the fluorescence FL detected by the fluorescence detection unit 9, and the generated fluorescent image 15a is output to the display device 30. With this, the physician in charge of the treatment can preform the treatment of the cancer treatment target site Pa by the treatment light TL while confirming the fluorescent image 15a representing the cancer treatment target site Pa displayed on the display device 30. As a result, the physician can easily perform the treatment of the cancer treatment target site Pa by the treatment light TL.

### Modified Embodiment

It should be noted that the embodiments disclosed herein are to be considered in all respects as illustrative and not restrictive. The scope of the present invention is indicated not by the embodiments described above but by claims and includes all modifications within the meanings and ranges equivalent to those of the claims.

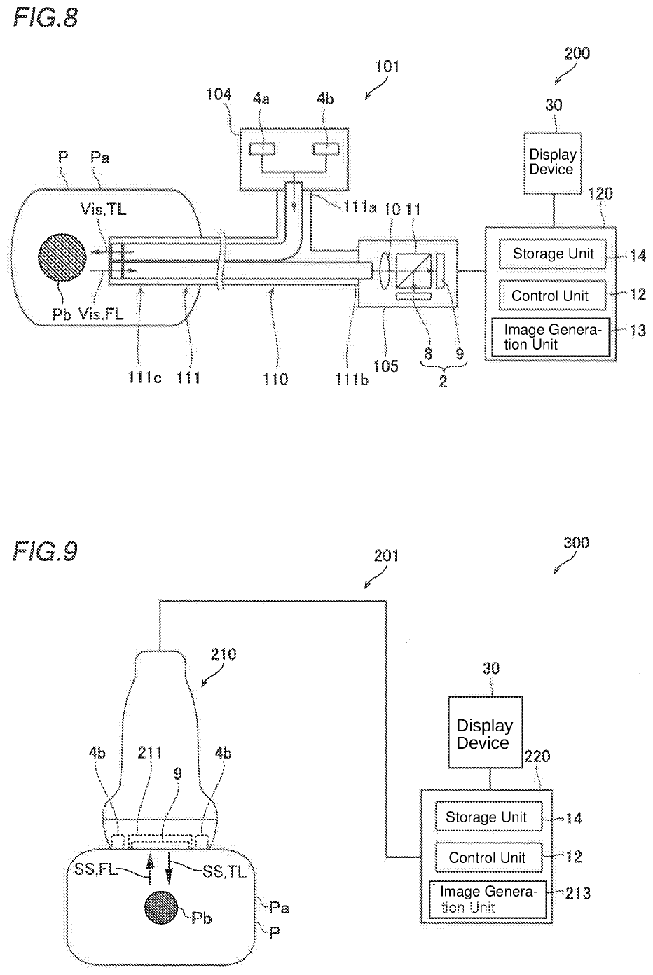

For example, in the above-described embodiment, an example is described in which the present invention is applied to a treatment support apparatus for supporting cancer treatment when performing cancer treatment by irradiating treatment light from the outside of a subject, but the present invention is not limited to this. The present invention may be applied, for example, as shown in FIG. 8, to a treatment support apparatus 101 for supporting cancer treatment when performing cancer treatment by irradiating the inside of a subject P with treatment light TL. Note that the same components as those of the above-described embodiment are denoted by the same reference numerals in the drawings, and the detailed descriptions thereof are omitted.

As shown in FIG. 8, the treatment support system 200 is provided with a treatment support apparatus 101 and a display device 30. The treatment support apparatus 101 is provided with an endoscope device 110 and a main body portion 120. The endoscope device 110 includes a light source unit 104 including a visible light source 4a and a treatment light source 4b, and an imaging unit 105 including a light receiving unit 2 and an optical system 3. The main body portion 120 includes a control unit 12, an image generation unit 13, and a storage unit 14. The endoscope device 110 includes a flexible and deformable cable unit 111.

In the cable unit 111, the branched root portions 111a and 111b are connected to the light source unit 104 and the imaging unit 105, respectively. In the cable unit 111, the distal end portion 111c is inserted into a subject P so as to reach the vicinity of the cancer treatment target site Pa inside the subject P. The cable unit 111 is configured to guide the visible light Vis irradiated from the visible light source 4a of the light source unit 104 and the treatment light TL irradiated from the treatment light source 4b of the light source unit 104 to the cancer treatment target site Pa. The cable unit 111 is configured to guide the fluorescence FL emitted from the photosensitizer Pb to the imaging unit 105 by being irradiated with the visible light Vis and the treatment light TL reflected by the cancer treatment target site Pa.

The detection of the fluorescence FL by the imaging unit 105, the determination of the cancer region by the control unit 12, the estimation of the treatment finish time by the control unit 12, and the determination of the treatment completion by the control unit 12 are the same as those of the embodiment described above.

Further, in the above-described embodiment, an example is described in which the present invention is applied to a treatment support apparatus for supporting cancer treatment when performing cancer treatment by emitting treatment light from a position apart from a subject, but the present invention is not limited to this. For example, as shown in FIG. 9, the present invention may be applied to a treatment support apparatus 201 for assisting cancer treatment when performing the cancer treatment by emitting treatment light TL from a position in contact with a skin of a subject P. Note that the same components as those of the above embodiment are denoted by the same reference numerals in the drawings and the detailed descriptions thereof are omitted.

As shown in FIG. 9, the treatment support system 300 is provided with a treatment support apparatus 201 and a display device 30. The treatment support apparatus 201 is provided with an ultrasonic probe unit 210 and a main body portion 220. The ultrasonic probe unit 210 includes a treatment light source 4b, a fluorescence detection unit 9, and an oscillator 211. The oscillator 211 includes a piezoelectric device and is configured to generate an ultrasonic wave SS and detect the ultrasonic wave SS.

The main body portion 220 includes a control unit 12, an image generation unit 213, and a storage unit 14. The ultrasonic probe unit 210 is used in a state in which the distal end portion is in contact with the skin of the subject P. The ultrasonic probe unit 210 emits the treatment light TL from the treatment light source 4b toward the treatment target site Pa and causes the oscillator to generate the ultrasonic wave SS towards the cancer treatment target site Pa, in a state in which the distal end portion is in contact with the skin of the treatment target site Pa of the subject P. In the ultrasonic probe unit 210, in a state in which the distal end portion is in contact with the skin of the cancer treatment target site Pa of the subject P, the fluorescence detection unit 9 detects the fluorescence FL emitted from the photosensitizer Pb by being irradiated with the treatment light TL and the oscillator 211 detects the ultrasonic wave SS reflected from the inside of the subject P.

The image generation unit 213 generates a fluorescent image 15a based on the fluorescence FL detected by the fluorescence detection unit 9 and generates an ultrasonic image of an inside of the subject P based on the ultrasonic wave SS detected by the oscillator 211. The determination of the cancer region by the control unit 12, the estimation of the treatment finish time by the control unit 12, and the determination of the treatment completion by the control unit 12 are the same as those in the embodiment described above.

In the above-described embodiment, an example is shown in which the treatment support apparatus is configured to detect the visible light and the fluorescence, but the present invention is not limited to this. In the present invention, the treatment support apparatus may be configured such that only the fluorescence can be detected without providing a visible light detection unit.

In the above-described embodiment, an example is shown in which the treatment support apparatus is configured to generate a fluorescent image, but the present invention is not limited to this. In the present invention, the treatment support apparatus is not necessarily required to be configured to generate a fluorescent image.

In the above-described embodiment, an example is shown in which the treatment support apparatus is configured to include a light source unit including the visible light source and the treatment light source, but the present invention is not limited thereto. In the present invention, the treatment support apparatus does not necessarily have to be configured to provide a light source unit including a visible light source and a treatment light source. In this case, a light source device dedicated to emit treatment light toward a subject can be used.

In the above-described embodiment, an example is shown in which the cancer region is determined based on the comparison of the temporal change value of the signal value of the fluorescence detected by the fluorescence detection unit with a threshold value, but the present invention is not limited to this. In the present invention, since the cancer signal waveform representing the temporal change of the signal value of the cancer-derived fluorescence and the non-cancer signal waveform representing the temporal change of the signal value of the non-cancer-derived fluorescence greatly differ from each other, the cancer region may be determined based on the waveform based on the temporal change of the signal value of the fluorescence detected by the fluorescence detection unit. Further, the cancer region may be determined based on the standard deviation of the signal value of the fluorescence detected by the fluorescence detection unit. In this case, for example, when the signal value of the fluorescence detected by the fluorescence detection unit deviates from the range of the standard deviation of the signal value of the fluorescence, it may be determined that the region is a cancer region.

In the above-described embodiment, an example is shown in which both the cancer region and the non-cancer region are determined, but the present invention is not limited to this. In the present invention, it may be configured such that only a cancer region is determined and a non-cancer region is not determined.

In the above-described embodiment, an example is shown in which the cancer region is determined based on the temporal change value of the signal value of the fluorescence detected by the fluorescence detection unit at the early stage of the irradiation period of the treatment light, but the present invention is not limited to this. For example, the cancer region may be determined based on the temporal change value of the signal value of the fluorescence detected by the fluorescence detection unit after the early stage of the irradiation period of the treatment light.

In the above-described embodiment, an example is shown in which the treatment support apparatus is configured to determine the cancer region and estimate the time when the treatment of the cancer treatment target site by the treatment light is finished, but the present invention is not limited to this. In the present invention, in cases where it is configured to determine the cancer region, it is not necessarily to be configured such that the treatment support apparatus estimates the time when the treatment of the cancer treatment target site by the treatment light is finished.

In the above-described embodiment, an example is shown in which the time when the treatment of the cancer treatment target site by the treatment light is finished is estimated based on the temporal change value of the signal value of the fluorescence detected by the fluorescence detection unit at the early stage of the irradiation period of the treatment light, but the present invention is not limited to this. For example, the time when the treatment of the cancer treatment target site by the treatment light ends may be estimated based on the temporal change value of the signal value of the fluorescence detected by the fluorescence detection unit after the early stage of the irradiation period of the treatment light.

In the above-described embodiment, an example is shown in which the function is configured to be a function representing a waveform having two peaks, but the present invention is not limited to this. In the present invention, it is not required to configure such that the function is a function representing a waveform having two peaks. In cases where the waveform of the signal value obtained from the cancer-derived fluorescence differs depending on the photosensitizer used for treatment, a function representing a waveform specific to the photosensitizer used for the treatment may be used.

In the above-described embodiment, an example is shown in which the function representing the decay curve according to the temporal change of the signal value of the fluorescence in the cancer region detected by the fluorescence detection unit is set to a Double-Boltzmann function, but the present invention is not limited to this. In the present invention, as long as the temporal change of the signal value of the fluorescence in the cancer region can be indicated, the function representing the decay curve may be a function, such as, e.g., an exponential function, a Sigmoid function, and a Boltzmann function, other than a Double-Boltzmann function. Further, for example, a function representing a decay curve may be a dedicated function.

In the above-described embodiment, an example is shown in which the treatment support apparatus is configured to determine the cancer region and whether to complete the treatment of the cancer treatment target site by the treatment light, but the present invention is not limited to this. In the present invention, as long as it is configured to determine the cancer region, it is not necessarily required such that the treatment support apparatus is configured to determine whether to complete the treatment of the cancer treatment target site by the treatment light.

1. A treatment support apparatus for supporting cancer treatment by imaging a cancer treatment target site of a subject when performing the cancer treatment by irradiating a photosensitizer accumulated in the cancer treatment target site with treatment light, the treatment support apparatus comprising:
a fluorescence detection unit configured to detect fluorescence emitted from the photosensitizer by being irradiated with the treatment light;
a determination unit configured to determine a cancer region based on a temporal change of a signal value of the fluorescence detected by the fluorescence detection unit.

2. The treatment support apparatus as recited in claim 1,
wherein the determination unit is configured to determine that a region is a cancer region when a temporal change value of the signal value of the fluorescence detected by the fluorescence detection unit is equal to or greater than a threshold value.

3. The treatment support apparatus as recited in claim 2,
wherein the determination unit is configured to determine that a region is a non-cancer region when the temporal change value of the signal value of the fluorescence detected by the fluorescence detection unit is less than the threshold value.

4. The treatment support apparatus as recited in claim 1,
wherein the determination unit is configured to determine the cancer region based on the temporal change of the signal value of the fluorescence detected by the fluorescence detection unit in a predetermined period after initiation of irradiation of the treatment light.

5. The treatment support apparatus as recited in claim 1, further comprising:
an estimation unit configured to estimate a finish time of treatment of the cancer treatment target site by the treatment light based on the temporal change of the signal value of the fluorescence in the cancer region detected by the fluorescence detection unit in a predetermined period after initiation of irradiation of the treatment light.

6. The treatment support apparatus as recited in claim 5,
wherein the estimation unit is configured to acquire a function representing the temporal change of the signal value of the fluorescence in the cancer region based on the temporal change of the signal value of the fluorescence in the cancer region detected by the fluorescence detection unit in the predetermined period and estimate the finish time of the treatment of the cancer treatment target site by the treatment light based on the acquired function.

7. The treatment support apparatus as recited in claim 6,
wherein the function is a function representing a waveform having two peaks.

8. The treatment support apparatus as recited in claim 7,
wherein the function includes a function representing a decay curve corresponding to the temporal change of the signal value of the fluorescence in the cancer region detected by the fluorescence detection unit.

9. The treatment support apparatus as recited in claim 4,
wherein the predetermined period is a period from the initiation of irradiation of the treatment light to a start of a decrease in the signal value of the fluorescence.

10. The treatment support apparatus as recited in claim 1,
wherein the determination unit is configured to determine whether to complete the treatment of the cancer treatment target site by the treatment light based on the temporal change of the signal value of the fluorescence in the cancer region detected by the fluorescence detection unit.

11. The treatment support apparatus as recited in claim 1,
wherein a fluorescent image is generated based on the fluorescence detected by the fluorescence detection unit, and the generated fluorescent image is output to a display unit.

12. A determination method in a treatment support apparatus for supporting cancer treatment by imaging a cancer treatment target site of a subject when performing the cancer treatment by irradiating a photosensitizer accumulated in the cancer treatment target site with treatment light, the determination method comprising the steps of:
detecting fluorescence emitted from the photosensitizer by being irradiated with the treatment light; and
determining a cancer region based on a temporal change of a signal value of the detected fluorescence.
